(19) United States
(12) Patent Application Publication
(10) Pub. No.: US 2009/0084987 A1
(43) Pub. Date: Apr. 2, 2009

(54) CHARGE NEUTRALIZATION IN A PLASMA PROCESSING APPARATUS

(75) Inventors: Ludovic GODET, Wakefield, MA (US); Svetlana RADOVANOV, Marblehead, MA (US); George D. PAPASOULIOTIS, North Andover, MA (US); Deven M. RAJ, Wenham, MA (US); Vikram SINGH, North Andover, MA (US); Timothy J. MILLER, Ipswich, MA (US); Ziwei FANG, Beverly, MA (US)

Correspondence Address: VARIAN SEMICONDUCTOR EQUIPMENT ASSC., INC., 35 DORY RD., GLOUCESTER, MA, 01930-2297, US

(73) Assignee: VARIAN SEMICONDUCTOR EQUIPMENT ASSOCIATES, INC., Gloucester, MA (US)

(21) Appl. No.: 11/863,728

(22) Filed: Sep. 28, 2007

(51) Int. Cl. A61N 5/00

(52) U.S. Cl. 250/492.3

(57) ABSTRACT

A plasma processing apparatus includes a process chamber, a source configured to generate a plasma in the process chamber, and a platen configured to support a workpiece in the process chamber. The platen is biased with a pulsed platen signal having pulse ON and OFF time periods to accelerate ions from the plasma towards the workpiece during the pulse ON time periods and not the pulse OFF time periods. A plate is positioned in the process chamber. The plate is biased with a plate signal to accelerate ions from the plasma towards the plate to cause an emission of secondary electrons from the plate during at least a portion of one of the pulse OFF time periods of the pulsed platen signal to at least partially neutralize charge accumulation on the workpiece.

### FIELD

This disclosure relates to plasma processing, and more particularly to charge neutralization in a plasma processing apparatus.

### BACKGROUND

A plasma processing apparatus generates a plasma in a process chamber for treating a workpiece supported by a platen in the process chamber. A plasma processing apparatus may include, but not be limited to, doping systems, etching systems, and deposition systems. The plasma processing apparatus may have a pulsed mode operation where the platen is biased with a pulsed platen signal having pulse ON and OFF time periods. Ions from the plasma are accelerated towards the workpiece during the pulse ON periods. As the ions strike the workpiece, charge may accumulate on the workpiece during the pulse ON periods.

In plasma doping systems continually having a plasma, any positive charge accumulation during the pulse ON periods tends to be efficiently neutralized during the pulse OFF periods by electrons in the plasma when the duty cycle of the pulsed platen signal is relatively low. However, there is a need to increase the duty cycle of the pulsed platen signal in order to increase throughput and maintain the doping levels required for some modern devices. For example, it is desirable to perform poly gate doping and counter doping of some state-of-the art devices by plasma doping with a duty cycle greater than 40%.

As the duty cycle of the pulsed platen signal increases above about 40%, there is a shorter period of time for neutralizing the charge accumulated on the workpiece during the pulse OFF periods. In addition, in plasma systems where plasma is not formed during the pulse OFF periods, there are no electrons to neutralize the charge accumulated. Hence, charge can accumulate in such systems even at relatively lower duty cycles of the pulsed platen signal. Consequently, excessive charge accumulation may occur in either system. This can result in the development of a relatively high potential on the workpiece that can cause doping non-uniformities, arcing, micro-loading, and device damage. For example, thin gate dielectrics can be easily damaged by excess charge build up.

Accordingly, there is a need to provide techniques for charge neutralization in a plasma processing apparatus which overcomes the above-described inadequacies and shortcomings.

### SUMMARY

According to a first aspect of the disclosure, a plasma processing apparatus is provided. The plasma processing apparatus includes a process chamber, a source configured to generate a plasma in the process chamber, a platen configured to support a workpiece in the process chamber, the platen being biased with a pulsed platen signal having pulse ON and OFF time periods to accelerate ions from the plasma towards the workpiece during the pulse ON time periods and not the pulse OFF time periods, and a plate positioned in the process chamber. The plate is biased with a plate signal to accelerate ions from the plasma towards the plate to cause an emission of secondary electrons from the plate during at least a portion of one of the pulse OFF time periods of the pulsed platen signal to at least partially neutralize charge accumulation on the workpiece.

According to another aspect of the disclosure, a method of controlling charge accumulation is provided. The method includes accelerating ions from a plasma in a process chamber towards a workpiece supported by a platen within the process chamber during pulse ON periods and not during pulse OFF periods of a pulsed platen signal provided to the platen, and accelerating ions from the plasma towards a plate during at least a portion of one of the pulse OFF periods of the pulsed platen signal to cause emission of secondary electrons from the plate to at least partially neutralize charge accumulation on the workpiece.

### BRIEF DESCRIPTION OF THE DRAWINGS

For a better understanding of the present disclosure, reference is made to the accompanying drawings, in which like elements are referenced with like numerals, and in which:

FIG. 1 is a block diagram of a plasma processing apparatus consistent with an embodiment of the disclosure;

FIG. 2 are plots of a pulsed platen signal and differing plate signals for the plasma processing apparatus of FIG. 1;

FIG. 3 is a plot of another plate signal for the plasma processing apparatus of FIG. 1; and

FIGS. 4-6 are schematic cross sectional views of differing embodiments of the plate of FIG. 1.

### DETAILED DESCRIPTION

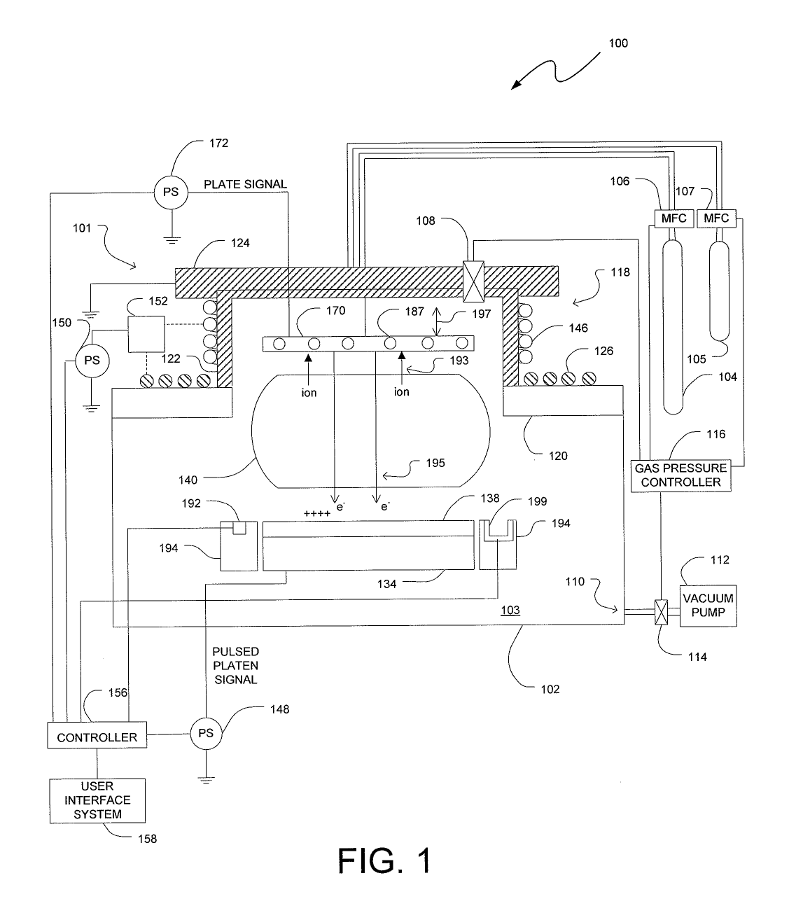

FIG. 1 is a block diagram of one plasma processing apparatus 100 having charge neutralization consistent with the present invention. In the embodiment of FIG. 1, the plasma processing apparatus 100 is a plasma doping system and will be described as such herein. The charge neutralization configuration described herein may also be utilized in other plasma processing apparatus including, but not limited to, etching and deposition systems where charge may accumulate on a workpiece. Furthermore, the plasma doping system of FIG. 1 is only one of many possible plasma doping systems that can perform ion implantation with charge neutralization according to the present invention.

The plasma doping system includes a process chamber 102 defining an enclosed volume 103. The process chamber 102 may be cooled or heated by a temperature regulation system (not illustrated). A platen 134 may be positioned in the process chamber 102 to support a workpiece 138. In one instance, the workpiece 138 may be a semiconductor wafer having a disk shape, e.g., a 300 millimeter (mm) diameter silicon wafer in one embodiment. The workpiece 138 may be clamped to a flat surface of the platen 134 by electrostatic or mechanical forces. In one embodiment, the platen 134 may include conductive pins (not shown) for making connection to the workpiece 138.

A gas source 104 provides a primary dopant gas to the interior volume 103 of the process chamber 102 through the mass flow controller 106. There may be a plurality of additional gas sources to provide a plurality of additional gases. In one instance, a secondary gas source 105 may provide a secondary gas to the interior volume 103 of the process chamber 102 through the mass flow controller 107.

A plate 170 is positioned in the process chamber 102. The plate 170 is biased to at least partially neutralize charge accumulation on the workpiece 138 during certain times. The plate 170 may also serve as a gas baffle to deflect the flow of gas from the gas sources 104 and 105. The plate 170 may also be movable in a direction perpendicular to the platen 134 as indicated by arrow 197. The plate 170 may have any desired shape and in one instance may have a disk shape. Although illustrated as having planar surfaces, the plate 170 may alternatively have arcuate or other shaped surfaces. Although the plate 170 is illustrated as being positioned directly above the workpiece 138, the plate 170 may be positioned in differing locations within the process chamber 102. The plate 170 may also optionally include a temperature regulation system to regulate the temperature of the plate 170. This may include passages 187 in the plate 170 to circulate fluid. The fluid may be cooling fluid or heating fluid.

A pressure gauge 108 measures the pressure inside the process chamber 102. A vacuum pump 112 evacuates exhausts from the process chamber 102 through an exhaust port 110 in the process chamber 102. An exhaust valve 114 controls the exhaust conductance through the exhaust port 110.

The plasma doping system may further includes a gas pressure controller 116 that is electrically connected to the mass flow controllers 106, 107, the pressure gauge 108, and the exhaust valve 114. The gas pressure controller 116 may be configured to maintain a desired pressure in the process chamber 102 by controlling either the exhaust conductance with the exhaust valve 114 or a process gas flow rate with the mass flow controller 106 in a feedback loop that is responsive to the pressure gauge 108.

The process chamber 102 may have a chamber top 118 that includes a first section 120 formed of a dielectric material that extends in a generally horizontal direction. The chamber top 118 also includes a second section 122 formed of a dielectric material that extends a height from the first section 120 in a generally vertical direction. The chamber top 118 further includes a lid 124 formed of an electrically and thermally conductive material that extends across the second section 122 in a horizontal direction. In some embodiments, the lid 124 may include a cooling system in order to dissipate a heat load generated during processing.

The plasma doping system may further include a source 101 configured to generate a plasma 140 within the process chamber 102. The source 101 may include a RF source 150 such as a power supply to supply RF power to either one or both of the planar antenna 126 and the helical antenna 146 to generate the plasma 140. The RF source 150 may be coupled to the antennas 126, 146 by an impedance matching network 152 that matches the output impedance of the RF source 150 to the impedance of the RF antennas 126, 146 in order to maximize the power transferred from the RF source 150 to the RF antennas 126, 146.

The plasma doping system may also include a bias power supply 148 electrically coupled to the platen 134. The bias power supply 148 is configured to provide a pulsed platen signal having pulse ON and OFF time periods to bias the platen 134, and hence the workpiece 138, to accelerate ions from the plasma 140 towards the workpiece 138 during the pulse ON time periods and not during the pulse OFF periods. The bias power supply 148 may be a DC or an RF power supply.

Another bias power supply 172 may be electrically coupled to the plate 170 to provide a plate signal to the plate 170. The plate 170 is biased with the plate signal to accelerate ions from the plasma 140 towards the plate 170 as indicated by arrows 193. Advantageously, ions striking the plate 170 cause an emission of secondary electrons (as illustrated by arrows 195) to at least partially neutralize a positive charge accumulation on the workpiece 138. Although illustrated as different power supplies, the power supplies 172 and 148, and even 150 may physically be the same power supply.

The plasma doping system may further include a charge monitor 192, a controller 156, and a user interface system 158. The charge monitor 192 may monitor charge accumulation or buildup and provide a charge signal representative of the charge accumulation on the workpiece 138 to the controller 156. The charge monitor 192 may be any type of charge monitor known in the art such as a capacitive type monitor. The charge monitor 192 may be positioned in a shield ring 194 proximate the workpiece 138. The shield ring 194 is disposed around the platen 134 in the embodiment of FIG. 1. As is known in the art, the shield ring 194 may be biased to improve the uniformity of implanted ion distribution near the edge of the workpiece 138. One or more Faraday sensors such as Faraday cup 199 may also be positioned in the shield ring 194 to sense ion beam current. The Faraday sensor may also include an annular Faraday sensor or segmented annular Faraday sensors positioned around the workpiece 138. The current level sensed by the Faraday sensor during the time when ions are accelerated towards the plate 170 is representative of the rate of secondary electron emission from the plate 170 and may be utilized by the controller 156 to monitor the actual rate of secondary electron emission. The controller 156 may adjust one or more parameters of the plate signal in response thereto to increase or decrease the rate of secondary electron emission.

The controller 156 can be or include a general-purpose computer or network of general-purpose computers that may be programmed to perform desired input/output functions. The controller 156 can also include other electronic circuitry or components, such as application specific integrated circuits, other hardwired or programmable electronic devices, discrete element circuits, etc. The controller 156 may also include communication devices, data storage devices, and software. For clarity of illustration, the controller 156 is illustrated as providing only an output signal to the power supplies 148, 150, 172 and receiving input signals from the charge monitor 192 and the Faraday cup 199. Those skilled in the art will recognize that the controller 156 may provide output signals to other components of the plasma doping system and receive input signals from the same. The user interface system 158 may include devices such as touch screens, keyboards, user pointing devices, displays, printers, etc. to allow a user to input commands and/or data and/or to monitor the plasma doping system via the controller 156.

In operation, the gas source 104 supplies a primary dopant gas containing a desired dopant for implantation into the workpiece 138. Examples of primary dopant gas include, but are not limited to, BF₃, BI₃, N₂, Ar, PH₃, AsH₃, B₂H₆, H₂, Xe, Kr, Ne, He, SiH₄, SiF₄, GeH₄, GeF₄, CH₄, CF₄, AsF₅, PF₃, and PF₅. The gas pressure controller 116 regulates the rate at which the primary dopant gas is supplied to the process chamber 102. The source 101 is configured to generate the plasma 140 within the process chamber 102. The source 101 may be controlled by the controller 156. To generate the plasma 140, the RF source 150 resonates RF currents in at least one of the RF antennas 126, 146 to produce an oscillating magnetic field. The oscillating magnetic field induces RF currents into the process chamber 102. The RF currents in the process chamber 102 excite and ionize the primary dopant gas to generate the plasma 140.

The secondary gas source 105 may also supply a secondary gas to the process chamber 102. The secondary gas may be an inert gas to have minimal effect on the doping process. The secondary gas may be a heavier gas than the primary dopant gas. In addition, the quantity of secondary gas provided may be relatively small compared to the quantity of primary dopant gas provided. The secondary gas may be selected to alter the emission of secondary electrons from the plate 170. For instance, some secondary gases may promote a greater amount of secondary electron emission with all other parameters being equal.

The bias power supply 148 provides a pulsed platen signal to bias the platen 134 and hence the workpiece 138 to accelerate ions from the plasma 140 towards the workpiece 138 during the pulse ON periods of the pulsed platen signal and not during the pulse OFF periods. The ions may be positively charged ions and hence the pulse ON periods of the pulsed platen signal may be negative voltage pulses with respect to the process chamber 102 to attract the positively charged ions. The frequency of the pulsed platen signal and/or the duty cycle of the pulses may be selected to provide a desired dose rate. The amplitude of the pulsed platen signal may be selected to provide a desired energy. Depending on the type of processing conditions, e.g., such as with relatively high duty cycles of the pulsed platen signal, excess charge can accumulate on the workpiece 138. An excess charge accumulation can result in the development of a relatively high potential on the workpiece 138 that can causing doping non-uniformities, arcing, micro-loading, and device damage.

Another bias power supply 172 provides a plate signal to bias the plate 170 to accelerate ions from the plasma 140 towards the plate 170 as illustrated by arrows 193. Ions striking the plate 170 cause an emission of secondary electrons as illustrated by arrows 195 to at least partially neutralize a positive charge accumulation on the workpiece 138. The emission of the secondary electrons from the plate 170 occurs during at least a portion of one of the pulse OFF time periods of the pulsed platen signal. An ancillary benefit of ions striking the plate 170 is that it tends to minimize the formation of a deposition layer on the plate 170. Therefore, maintenance frequency for the plate 170 can be reduced compared to a plate that is not struck by ions. In addition, better particle performance and process control can be achieved compared to a plate that is not struck by ions.

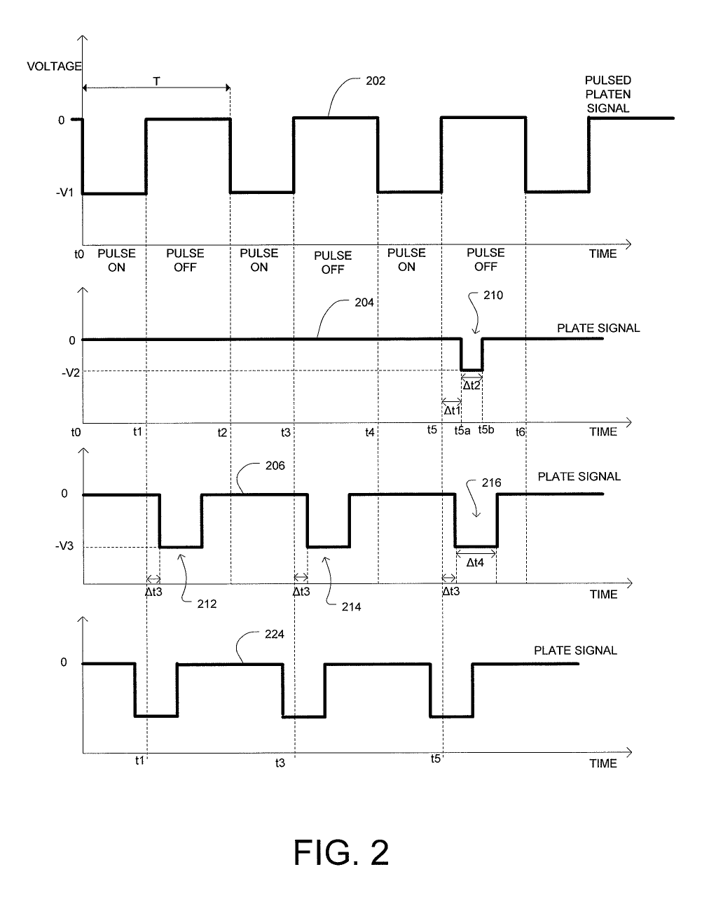

Turning to FIG. 2, a plot of an exemplary pulsed platen signal 202 is illustrated. In this instance, the pulsed platen signal 202 is a pulsed DC signal having a period T defining a frequency. A typical frequency may range between 100 Hz and 10 kHz. The pulsed platen signal 202 has alternating pulse ON and OFF time periods. For example, pulse ON time periods occur between time t0 and t1, t2 and t3, and so on, while pulse OFF time periods occur between times t1 and t2, t3 and t4, and so on. The duty cycle of the pulsed platen signal 202 is given by a ratio of the pulse ON time period to the period T. Therefore, a higher duty cycle results in shorter pulse OFF time periods. The pulsed platen signal 202 has a negative amplitude (−V1) with respect to the process chamber 102 during the pulse ON time periods to accelerate positive ions from the plasma 140 towards the workpiece 138. During the pulse ON time periods, excess charge may accumulate on the workpiece 138.

Differing parameters of the plate signal to bias the plate 170 can be varied to vary the quantity of secondary electron emission from the plate 170. These parameters may include voltage amplitude, pulse width, quantity of pulses, etc. In general, increasing the voltage amplitude would increase the yield of secondary electrons. Increasing the pulse width and the quantity of pulses would also generally increase the yield of secondary electrons with all other parameters being equal.

Several differing plate signals are illustrated in FIG. 2 to further illustrate how varying parameters of the plate signal can vary secondary electron emission from the plate 170. A first exemplary plate signal 204 is illustrated on a time axis coincident with the pulsed platen signal 202. As shown, the plate signal 204 is a pulsed DC signal that has a pulse ON time period 210 during a portion of one pulse OFF time period of the pulsed platen signal 202, e.g., in this instance during the pulse OFF time period between times t5 and t6. Although illustrated as a pulsed DC signal, those skilled in the art will recognize that the plate signal 204 may also be a pulsed RF signal. During the pulse ON time period 210, ions from the plasma 140 are accelerated towards the plate 170 to cause emission of secondary electrons. The pulse ON time period 210 has a start time (t5a) and stop time (t5b) defining a pulse width (Δt2). The start time (t5a) may be synchronized to start within a particular time interval (Δt1) of the end of the previous pulse ON time interval of the pulsed platen signal 202. In one embodiment, this particular time interval (Δt1) may be 0.1 microseconds. The start time (t5a) may also be coincident with the end of the previous pulse ON time interval of the pulsed platen signal 202. The number of pulse ON time periods, including the start time (t5a), stop time (t5b), and pulse width (Δt2) of each pulse ON period may be selected to provide a desired amount of secondary electron emission from the plate 170. Such parameters may be adjusted in response to an expected charge accumulation for a particular process on the workpiece 138 or a measured condition representative of actual charge accumulation.

A second exemplary plate signal 206 is also illustrated in FIG. 2. Similarly to the first plate signal 204, the second plate signal 206 is also a pulsed DC signal. Compared to the first pulsed plate signal 204, the second pulsed plate signal 206 is configured to bias the plate 170 to accelerate ions towards the plate during each pulse OFF time period of the pulsed platen signal 202. For example, the first pulse ON period 212 is synchronized to occur during the first pulse OFF period of the pulsed platen signal 202 between times t1 and t2. Similarly, the other pulse ON periods 214, 216 are synchronized to occur during the other pulse OFF periods of the pulsed platen signal 202. Compared to the first plate signal 204, the second plate signal 206 may result in the emission of more secondary electrons to at least partially neutralize a relatively greater expected or measured charge accumulation. The pulse ON periods 212, 214, and 216 may be synchronized to start within a particular time interval (Δt3) of an end of the previous pulse ON period periods of the pulsed platen signal 202. In one embodiment, this particular time interval (Δt3) may be 0.1 microseconds. Parameters such as the pulse width (Δt4) and amplitude (−V3) of the signal 206 may also be varied to control the yield of secondary electrons emitted from the plate 170.

A third exemplary plate signal 224 is also illustrated in FIG. 2. Compared to the second plate signal 206, the third plate signal may have pulse ON periods that start slightly before the start of the pulse OFF periods of the pulsed platen signal 202, and continue for at least a portion of the pulse OFF periods.

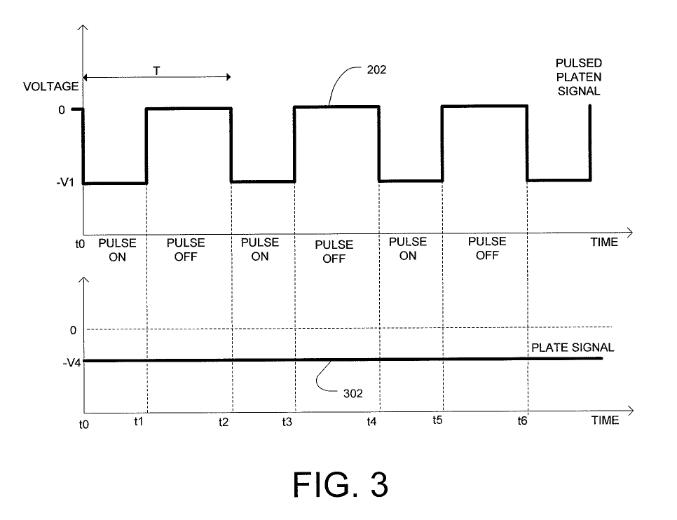

Turning to FIG. 3, yet another plot of a plate signal 302 is illustrated on a time axis coincident with the pulsed platen signal 202 of FIG. 2. Compared to the plate signals of FIG. 2, the plate signal 302 is a constant negative voltage (−V4) with respect to the process chamber 102 to continuously accelerate ions from the plasma 140 towards the plate 170 during both the pulse ON and OFF time periods of the pulsed platen signal. The voltage amplitude (V4) is selected to be much less than the amplitude of the pulsed platen signal (V4<<V1). In this way, ions will still be accelerated to the workpiece 138 during the pulse ON time periods of the pulsed platen signal 202. By controlling the amplitude (V4) of the plate signal 302, the rate of acceleration of ions from the plasma towards the plate 170 is controllable to control a plasma density of the plasma 140 during the pulse ON time periods of the pulsed platen signal 202. In general, a relatively higher plasma density can be achieved during the pulse ON time periods with the plate signal 302 than compared to the plate signals 204 and 206 given the greater number of ionizing collisions between electrons and gas molecules of the process gas.

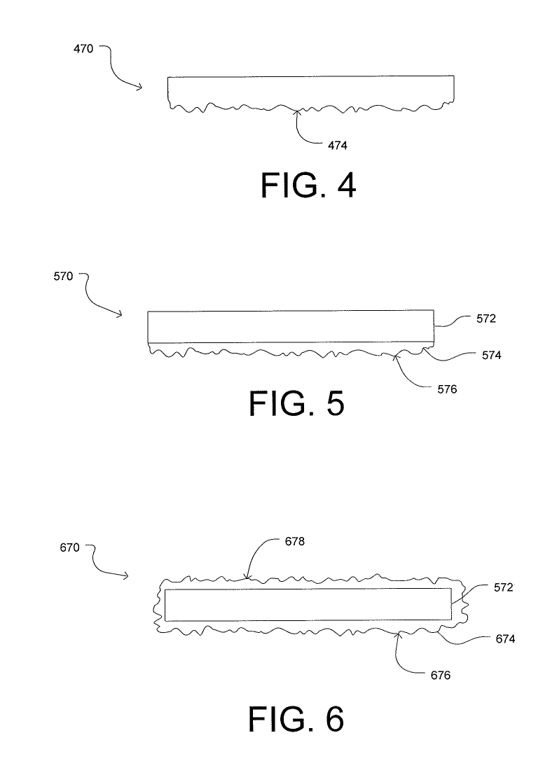

Turning to FIGS. 4 to 6, schematic cross sectional views of differing embodiments of plates consistent with the invention are illustrated. The plates 470, 570, 670 may have a variety of geometries and in one instance are disk shaped to match a workpiece 138 that may also be disk shaped. Plate materials may be selected as required to increase or decrease the secondary electron yield.

FIG. 4 illustrates a plate 470 having a roughened surface 474 facing the workpiece 138 to promote the emission of secondary electrons. The roughened surface 474 provides a larger surface area compared to a polished surface to provide for relatively more collisions of ions with the surface 474.

FIG. 5 is a schematic cross sectional view of another embodiment where the plate 570 may be fabricated of a conductor 572 coated on a surface of the conductor 572 facing the workpiece 138 with a silicon film 574. The conductor 572 may include, but not be limited to, aluminum and nickel. The silicon film 574 may also have a roughened surface 576 facing the workpiece.

FIG. 6 is a schematic cross sectional view of yet another embodiment of a plate 670 that may also be fabricated of the conductor 572. Compared to the embodiment of FIG. 5, the silicon film 674 is disposed around the entire exterior surface of the conductor 572. In this way, the emission of secondary electrons is promoted by the roughened surface 676 facing the workpiece and encapsulating the entire conductor 572 avoids any metal contamination from the conductor 572.

Accordingly, there is provided a charge neutralization apparatus and method to at least partially neutralize charge accumulation on a workpiece of a plasma processing apparatus. The duty cycle of the pulsed platen signal that accelerates ions towards the workpiece can therefore be increased without creating excessive charge accumulation. Excessive charge accumulation in a plasma doping system can lead to doping non-uniformities, arcing, and device damage. In addition, this charge neutralization apparatus and method is particularly useful for plasma systems that generate plasma only during certain time intervals. This is because such systems do not have plasma and hence electrons in the plasma to assist with charge neutralization efforts during other time intervals.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A plasma processing apparatus comprising:
a process chamber;
a source configured to generate a plasma in the process chamber;
a platen configured to support a workpiece in the process chamber, the platen being biased with a pulsed platen signal having pulse ON and OFF time periods to accelerate ions from the plasma towards the workpiece during the pulse ON time periods and not the pulse OFF time periods; and
a plate positioned in the process chamber, the plate being biased with a plate signal to accelerate ions from the plasma towards the plate to cause an emission of secondary electrons from the plate during at least a portion of one of the pulse OFF time periods of the pulsed platen signal to at least partially neutralize charge accumulation on the workpiece.

2. The plasma processing apparatus of claim 1, wherein the plate has a roughened surface to promote the emission of secondary electrons.

3. The plasma processing apparatus of claim 1, wherein the plate has a disk shape.

4. The plasma processing apparatus of claim 1, wherein the plate comprises a conductor coated on at least a surface of the conductor with a silicon film, the silicon film having a roughened surface to promote the emission of secondary electrons.

5. The plasma processing apparatus of claim 1, wherein the plate is biased with the plate signal to accelerate ions from the plasma towards the plate during at least a portion of each of the pulse OFF periods of the pulsed platen signal.

6. The plasma processing apparatus of claim 1, wherein the plate signal is a pulsed plate signal having pulse ON and OFF time periods to accelerate ions from the plasma towards the plate during the pulse ON time periods and not the pulse OFF time periods, and wherein pulse ON periods of the pulsed plate signal are synchronized to occur during the pulse OFF periods of the pulsed platen signal.

7. The plasma processing apparatus of claim 6, wherein the pulse ON periods of the pulsed plate signal are synchronized to start within 0.1 microseconds of an end of the pulse ON periods of the pulsed platen signal.

8. The plasma processing apparatus of claim 1, wherein the plate is biased with the plate signal to continuously accelerate ions from the plasma towards the plate during the pulse ON and OFF periods of the pulsed platen signal.

9. The plasma processing apparatus of claim 8, wherein a rate of acceleration of ions from the plasma towards the plate is controllable to control a plasma density of the plasma during the pulse ON periods of the pulsed platen signal.

10. The plasma processing apparatus of claim 1, further comprising a charge monitor configured to provide a charge monitor signal representative of charge accumulation on the workpiece, and a controller responsive to the charge monitor signal to control the plate bias signal in response to the charge monitor signal.

11. The plasma processing apparatus of claim 1, further comprising a primary gas source configured to provide a primary dopant gas into the process chamber and a secondary gas source configured to provide a secondary gas into the process chamber, wherein the secondary gas is selected to alter the emission of secondary electrons from the plate.

12. A method of controlling charge accumulation comprising:
accelerating ions from a plasma in a process chamber towards a workpiece supported by a platen within the process chamber during pulse ON periods and not during pulse OFF periods of a pulsed platen signal provided to the platen; and
accelerating ions from the plasma towards a plate during at least a portion of one of the pulse OFF periods of the pulsed platen signal to cause emission of secondary electrons from the plate to at least partially neutralize charge accumulation on the workpiece.

13. The method of claim 12, wherein the accelerating ions from the plasma towards the plate occurs during at least a portion of each of the pulse OFF periods of the pulsed platen signal.

14. The method of claim 13, wherein the accelerating ions from the plasma towards the plate is synchronized to start within a selected time period of each of the pulse ON periods of the pulsed plasma signal.

15. The method of claim 12, wherein the accelerating ions from the plasma towards the plate occurs continuously during the pulse ON and OFF periods of the pulsed plasma signal.

16. The method of claim 15, further comprising controlling a rate of acceleration of the ions from the plasma towards the plate to control a plasma density of the plasma during the pulse ON periods of the pulsed platen signal.

17. The method of claim 12, further comprising monitoring a condition representative of charge accumulation on the workpiece, and controlling the accelerating of ions from the plasma towards the plate in response to the monitored condition.

18. The method of claim 12, further comprising providing a primary dopant gas and a secondary gas into the process chamber, wherein the secondary gas is selected to alter the emission of secondary electrons from the plate.
